(19) United States
(12) Patent Application Publication
(10) Pub. No.: US 2026/0212978 A1
(43) Pub. Date: Jul. 23, 2026

(54) METHOD AND/OR SYSTEM FOR CREATING A STRUCTURED REPORT

(71) Applicant: KONINKLIJKE PHILIPS N.V., EINDHOVEN (NL)

(72) Inventors: Fabian WENZEL, HAMBURG (DE); Nick FLÄSCHNER, HAMBURG (DE); Arne EWALD, HAMBURG (DE); Nikita MARKOV, MOSKVA (RU); Mikhail PADALKO, VELDHOVEN (NL); John Cornelius Petrus VAN DER VEN, HELMOND (NL)

(21) Appl. No.: 19/139,445

(22) PCT Filed: Dec. 13, 2023
(86) PCT No.: PCT/EP2023/085461

§ 371 Date: Jun. 16, 2025

(30) Foreign Application Priority Data
Dec. 22, 2022 (RU) 2022133857

(51) Int. Cl. G16H 15/00

(52) U.S. Cl. CPC G16H 15/00

(57) ABSTRACT

A computer-implemented method is configured to receive machine learning finding results from an evaluation of imaging data for a subject for at least a subset of editable report fields of an electronic structured report for the subject from a remote device configured to perform the evaluation, and rank the machine learning finding results based on predetermined criteria. A system (102) includes a computer readable storage medium (108) and a processor (106). The computer readable storage medium includes computer readable instructions. The processor is configured to execute the computer readable instructions, which causes the processor to receive machine learning finding results from an evaluation of imaging data for a subject for at least a subset of editable report fields (202) of an electronic structured report for the subject from a remote device configured to perform the evaluation, and rank the machine learning finding results based on predetermined criteria.

### TECHNICAL FIELD

The following generally relates to medical imaging reports and more particularly to creating a structured medical imaging report, and is amenable to creating other structured reports.

### BACKGROUND

Example workflow for a radiology examination includes a referring clinician prescribing an imaging examination of a subject via an imaging order, a radiology department/center performing the imaging examination of the subject in accordance with the imaging order, the radiology department/center creating a report based on findings at least from a radiologist's interpretation of an image from the imaging examination, and the radiology department/center providing the referring clinician with access to the report.

Such reports can be unstructured or structured. An example of an unstructured report is a report generated by a radiologist dictating free text into a recording device and a transcriptionist transcribing the recording to generate the report. In another example, transcription (speech-to-text) software is utilized to transcribe the free text of the radiologist into the report. An example of a structured report is a digital form with editable report fields that are to be filled in with entries from predetermined lists of entries for the fields.

With unstructured reports, different clinicians describe what is imaged using different language in different ways to describe the same thing. Structured reports in radiology can lead to a more standardized and thus quality-controlled reporting from the radiologist to the referring clinician, which in turn will lead to better informed treatment decisions and thus better patient outcomes. However, it is not readily clear how structured reports can be timely completed when the input to the report system includes uncertain findings.

As such, there is an unresolved need for an improved approach(s) for creating a structured report.

### SUMMARY

Aspects described herein address the above-referenced problems and/or others.

In one aspect, a computer-implemented method is configured to receive machine learning finding results from an evaluation of imaging data for a subject for at least a subset of editable report fields of an electronic structured report for the subject from a remote device configured to perform the evaluation, and rank the machine learning finding results based on predetermined criteria.

In another aspect, a system includes a computer readable storage medium with computer readable instructions, a processor configured to execute the computer readable instructions, which causes the processor to receive machine learning finding results from an evaluation of imaging data for a subject for at least a subset of editable report fields of an electronic structured report for the subject from a remote device configured to perform the evaluation, and rank the machine learning finding results based on predetermined criteria.

In another aspect, a computer readable storage medium stores computer readable instructions, which when executed by a processor of a computer cause the processor to receive machine learning finding results from an evaluation of imaging data for a subject for at least a subset of editable report fields of an electronic structured report for the subject from a remote device configured to perform the evaluation, wherein the machine learning finding results include classification results with corresponding classification probabilities or regression values with corresponding confidence intervals, and rank the machine learning finding results based on predetermined criteria.

Those skilled in the art will recognize still other aspects of the present application upon reading and understanding the attached description.

### BRIEF DESCRIPTION OF THE DRAWINGS

The invention may take form in various components and arrangements of components, and in various steps and arrangements of steps. The drawings are only for purpose of illustrating the embodiments and are not to be construed as limiting the invention.

FIG. 1 diagrammatically illustrates an example system, in accordance with an embodiment(s) herein.

FIG. 2 illustrates an example of an electronic structured report with unpopulated editable report fields.

FIG. 3 illustrates an example of the electronic structured report of FIG. 2 with the editable report fields populated with entries.

FIG. 4 illustrates an example method for manually populating editable report fields of an electronic structured report where the input includes classification results and classification probabilities, in accordance with an embodiment(s) herein.

FIG. 5 illustrates an example method for manually populating editable report fields of an electronic structured report where the input includes regression values and confidence intervals, in accordance with an embodiment(s) herein.

FIG. 6 illustrates an example method for automatically populating editable report fields of an electronic structured report where the input includes classification results and classification probabilities, in accordance with an embodiment(s) herein.

FIG. 7 illustrates an example method for automatically populating editable report fields of an electronic structured report where the input includes regression values and confidence intervals, in accordance with an embodiment(s) herein.

### DESCRIPTION OF EMBODIMENTS

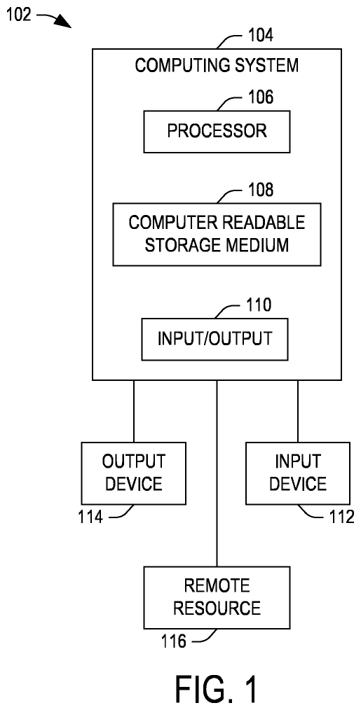

FIG. 1 diagrammatically illustrates an example system 102.

The system 102 includes a computing system 104, such as a computer, a workstation, etc. The computing system 104 includes a processor 106 and computer readable storage medium 108. Non-limiting examples of suitable processors include a central processing unit (CPU), a microprocessor (μP), a graphics processing unit (GPU), and/or other processor. The computer readable storage medium 108 includes non-transitory storage medium such as physical memory, a memory device, etc., and excludes transitory medium. The computing system 104 further includes input/output (I/O) 110. The computing system 104 can be part of a picture archiving and communication system (PACS), an advanced visualization system for radiologists such as Philips® Intellispace® Portal, a Cardiovascular Information System (CVIS) such as Philips® Intellispace® Cardiovascular or Cardiology PACS (C-PACS), a computer workstation, a server, and/or other specialized apparatus for radiology or cardiology workflow or reading images.

An input device 112 is in electrical communication with the computing system 104 via the I/O 110. A non-limiting example of the input device 112 includes a keyboard, a mouse, a microphone, etc. The input device 112 includes one or more input devices. An output device 114 is also in electrical communication with the computing system 104 via the I/O 110. A non-limiting example of the output device 114 includes a display monitor, a speaker, etc. The output device 114 includes one or more output devices. In one instance, the input device 112 and the output device 114 are separate devices (e.g., a keyboard and a display monitor). In another instance, the input device 112 and the output device 114 are the same device (e.g., a touch-screen monitor).

In the illustrated embodiment, a remote resource 116 is also in communication with the computing system 104 via the I/O 110. The remote resource 116 includes one or more remote resources. Non-limiting examples of the remote resource 116 include an imaging system, a computing and/or archival system, and/or other resources. Non-limiting examples of the imaging system include a magnetic resonance imaging (MRI), a computed tomography (CT), an X-ray, etc. system. Non-limiting examples of the computing and/or archival system includes cloud processing resources, a server, a workstation, a Radiology Information System (RIS), Hospital Information System (HIS), an electronic medical record (EMR), a PACS, and/or other computing and/or archival system.

The processor 106 is configured to execute a computer readable instruction encoded or embedded in the computer readable storage medium 108. At least one computer readable instruction, when executed by the processor 106, causes the processor 106 to evaluate input findings received from the remote resource 116 and present editable report fields for acquiring data to construct an electronic structured report, where the editable report fields are presented in a user interface (UI) displayed via a display monitor or the like of the output device 114. In one instance, the electronic structured report is compliant with the Digital Imaging and Communications in Medicine (DICOM) standard.

In one instance, the input findings from the remote resource 116 were determined based on a machine learning (e.g., artificial intelligence) algorithm(s). For example, in one instance the input at least includes findings from the remote resource 116 in the form of machine learning classification results. Additionally, or alternatively, the input at least includes findings from the remote resource 116 in the form of machine learning regression values. For example, in one instance the remote resource 116 utilizes a neural network(s), such as a convolutional neural network, a recurrent neural network, etc. that was trained on a set of images with findings where the output of the neural network(s) includes classification categorical labels and/or regression continuous values. In some instances, the input includes other information such as statistical information and/or other information. Furthermore, the input includes input determined based on one or more different models.

With such machine learning based input, the editable report fields are configured to receive the classification categorical labels and/or the regression continuous values. As described in greater detail below, the processor 106 processes the input and facilitates populating the editable report fields automatically (i.e. not based on a user input selecting an entry via the input device 112) and/or manually based on a user input selecting an entry via the input device 112. In one instance, this approach improves reporting systems where the machine learning input includes uncertain results, e.g., results with no clear correct result. The amount of time to complete the report may be more, the same or less than the time it takes to complete an unstructured report.

The following describes an example of an electronic structured report in connection with a knee imaging examination. This example is for explanatory purposes and is not limiting; other structured reports for the knee and/or other anatomy such as the heart, etc. are contemplated herein.

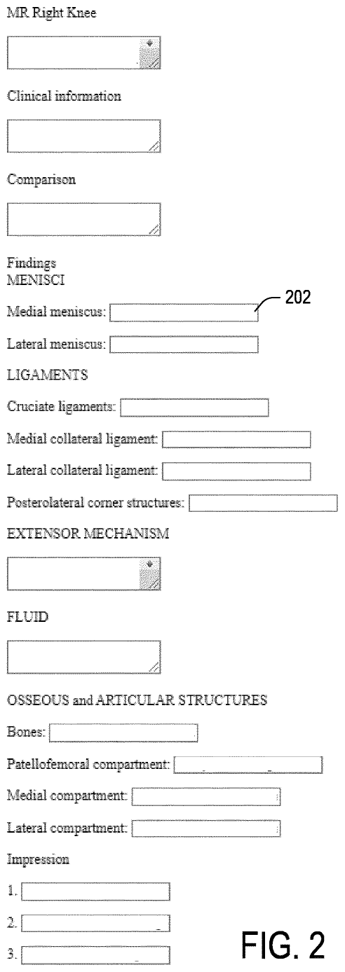

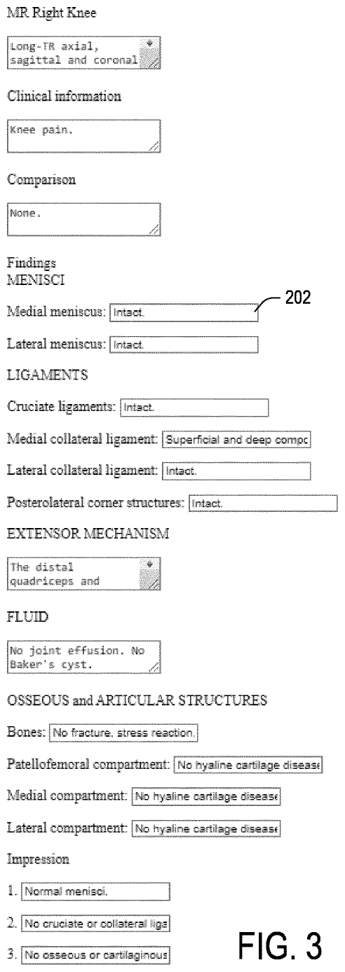

Briefly turning to FIGS. 2 and 3, an example of a structured report for an MRI of a knee is illustrated. The template is from the RadReport Template library, accessed at https://radreport.org/home for free templates not subject to licensing restrictions on their use. Other templates for the knee and/or other anatomy are also contemplated herein. FIG. 2 shows the structured report unpopulated in that none of the editable report fields (denoted by rectangular boxes) include an entry, and FIG. 3 shows the structured report populated in that all of the editable report fields include an entry.

For example, in FIG. 2, an editable report field 202 for the “Medial meniscus” under “Findings” and “MENISCI” is not populated with an entry, whereas in FIG. 3 the editable report field 202 is populated with the entry “Intact” (i.e., “Normal”). Generally, an editable report field is populated with an entry from a set of predetermined entries for the editable report field. For example, for the editable report field 202, the set of predetermined entries may include “Intact,” “Grade 1 tear,” “Grade 2 tear,” “Grade 3 tear,” etc. The editable report field 202 is populated manually via a user input selecting an entry from the set of predetermined entries, automatically by the system, or a combination thereof.

An example of a knee grading scheme includes the grading scheme for osteoarthritis severity grades (cartilage) by the International Cartilage Repair Society (ICRS). With this grading, “Grade 0” refers to normal cartilage (i.e. “Intact”), “Grade 1” refers to near-normal cartilage with superficial lesions, “Grade 2” refers to cartilage with lesions extending to less than 50% of the depth of the cartilage, “Grade 3” refers to cartilage with defects that extend to more than 50% of the depth of the cartilage, and “Grade 4” refers to severely abnormal cartilage where the cartilage defect reaches to subchondral bone. Other grading schemes are also contemplated herein.

The editable report field 202 in FIGS. 2 and 3 is configured to receive a categorical label, e.g., a classification result (e.g., “Intact”). In another instance, a structured report template additionally or alternatively includes an editable report field that is configured to receive a continuous value, e.g., a regression result (e.g., “TT-TG distance=5 mm”). In yet another instance, a structured report template additionally or alternatively includes an editable report field that is configured to receive an entry other than a categorical label or a continuous value. The illustrated structured report may be more or less table like, but other formats such as sentences are contemplated herein.

The processor 106 executes computer readable instruction in the computer readable storage medium 108, which causes the processor 106 to process the input classification results based on their respective class probabilities. By way of non-limiting example, where the input related to the editable report field 202 for the “Medial meniscus” includes a class “Intact” with a probability of 0.55 (p=0.55), a class “Grade 1 tear” with a probability of 0.25, a class “Grade 2 tear” with a probability of 0.15, and a class “Grade 3 tear” with a probability of 0.05, the processor 106 utilizes the class probabilities to rank/sort the classification results.

In one instance, the processor 106 ranks/sorts the classification results in descending order (from highest probability to lowest probability) based the class probabilities. For example and continuing with the above example, after the ranking/sorting, the class “Intact” would be first, the class “Grade 1 tear” would be second, the class “Grade 2 tear” would be third, and the class “Grade 3 tear” would be fourth since 0.55 is greater than 0.25, 0.25 is greater than 0.15, and 0.15 is greater than 0.05. In one instance this improves workflow as the most likely finding is always displayed at the top.

In one instance, the processor 106 presents the classification results in the UI in connection with the editable report field 202. For example, the classification results can be presented in a drop-down menu, a pop-up menu, a list box or the like, sorted, e.g., in descending order from greatest probability value to least probability value. For example, the classification results can be displayed as: “Intact,” “Grade 1 tear,” “Grade 2 tear” and “Grade 3 tear.” In another example, the probabilities are visually displayed or presented with their respective classification results to a user via a display monitor for example. With the classification results being currently presented to the user, the user selects only one, although the user can change which result is currently selected.

In response to user selection of a classification result presented in the UI (e.g., the computing system 104 receives an input from the input device 112), the processor 106 populates the editable report field 202 with the selected classification result. In this example of FIGS. 2 and 3, the user selected “Intact” from the displayed set of entries for the editable report field 202, and the processor 106 populated the editable report field 202 with the classification result “Intact.” Again, the user can change their selection via the drop-down menu, the pop-up menu, the list box or the like.

Once the editable report fields are populated, the electronic structured radiology report for the examination can be saved, printed, electronically sent to an entity (e.g., the subject, a referring clinician, a healthcare facility, etc.), etc.

The above example describes a structured report with editable report fields configured to receive classification results. For a structured report with editable report fields configured to receive regression results, the input includes regression results instead of classification results. Where a regression result does not include a confidence interval, the user can accept the result or reject the result and input a value manually. Where the regression results include confidence intervals, multiple values within that confidence interval can be displayed, e.g., ranked or sorted according to their deviation from the predicted value. Again, this improves workflow as the most likely finding is displayed at the top.

The following describes an example use case scenario where the computing system 104 is part of a PACS. After an imaging examination, the imaging data is uploaded to the PACS. The PACS provides a copy of the imaging data to one or more of the remote resources 116 for evaluation via one or more machine learning algorithms, as described herein, e.g., utilizing a trained neural network(s) that outputs classification categorical labels and/or regression continuous values as results. The results of the one or more machine learning algorithms are provided to the PACS. The imaging data is added to a worklist of a reading clinician, who is notified that the imaging data is accessible to be evaluated. The clinician utilizes the PACS to evaluate the imaging data and populate the editable report fields of an electronic structured report to create an electronic structured report, as described herein.

In one instance, the classification results displayed in the UI are visibly highlighted. For example, the classification results can be displayed with a color coding, e.g., the most probable class with green highlighting, the next most probable class with yellow highlighting, the next most probable class with orange highlighting, the next most probable class with red highlighting, etc. Other color coding schemes are also contemplated herein. For regression results, the color coding can reflect a distance from the predicted regression result and/or other information. Other highlighting is also contemplated herein. For example, suitable highlighting further includes, but is not limited to, shading, font size, optical, audio and/or other highlighting.

In another instance, the computer readable instruction causes the processor 106 to automatically select classification results for which a largest class probability exceeds a user-defined threshold (classification results input) or automatically selects a regression value for which a confidence interval subceeds a user-defined threshold value (regression values input). In this instance, the structured editable report fields with such classification results are populated without user input selecting a displayed result. However, the user can override an auto-populated entry by selecting a different result from the displayed results or manually entering an entry. For editable report fields where the input results do not satisfy their respective thresholds, the manual selection and entry described above and/or other approach is utilized.

In another instance, where an editable report field is automatically populated with a “normal” result (i.e. no finding), the processor 106 does not present a list of possible “abnormal” results for user selection, but moves on to the next editable report field, essentially “skipping” over the editable report field such that the user would only interact with editable report fields with likely abnormal findings. However, the user can override an automatically populated entry of “normal” and cause the results to be displayed and select one of the displayed results to populate the editable report field, as described herein. The user can also activate the editable report field to cause the results to be displayed even where the automatically populated results satisfies the user.

In another instance, where an editable report field for a first anatomical structure is likely normal and another editable report field for another structure in close proximity to the first anatomical structure is likely an abnormal result, the editable report field for the first anatomical structure is not “skipped.” For example, when the anterior horn of the lateral meniscus is classified as 99.9% normal but the neighboring pars intermedia of the lateral meniscus is classified as 99.9% abnormal, the editable report fields are not automatically populated or “skipped,” and the user can manually populate the field as described herein and/or otherwise.

In another instance, where a set of anatomical sub-structures can be grouped into a larger anatomical structure (i.e. the base and apex of the patella form the patella), all sub-structures and accordingly the larger structure are automatically classified as “normal” when all smaller sub-structures normal class probabilities exceed a threshold which may be lower than for individual automatic classifications. For example, when any structure is automatically classified as “normal” when the normal class probability of 80% is exceeded, the patella and accordingly the base and apex of the patella could be also automatically classified as “normal” as long as all sub-structures (apex and base) “normal” class probabilities exceed a lower threshold, e.g., 75%. It is to be appreciated that the threshold percentages in this example are provided for explanatory purpose and can be different. In another instance, the computer readable instructions cause the processor 106 to automatically reject classification results for which the largest class probability subceeds a user-defined threshold value or the confidence interval exceeds a user-defined threshold value such that only “rather likely” classes/regression results are displayed to the user and available for selection to populate an editable report field.

In another instance, the computer readable instructions cause the processor 106 to automatically reject all classification results when a user-defined entropy of the class probabilities is exceeded (i.e., for a flat classification probability distribution, no suggestions are made). A suitable approach for computing an entropy includes H(X)=−Σᵢp(i)logp(i) where H is the computed entropy, X is the classification result with N entries (one per possible class) and p(i) denotes the probability for class i, with i,=1, . . . , N . . . .

In another instance, the computer readable instructions cause the processor 106 to only display a user-specified number of possible entries (i.e. only the 3 most likely classification results).

In another instance, the input to the computing system 104 further includes a result from a statistical model (i.e. age-dependent likelihoods of diseases etc.), and the sorted/ranked results can not only depend on the classification probabilities or regression confidence intervals but also on the already confirmed by the radiologist (e.g., when a new rupture of the anterior cruciate ligament is confirmed by the radiologist, the often statistically correlated likelihood of a finding of a bone bruise is ranked higher by the recommender system than when no cruciate ligament rupture is present.). As described herein, in one instance the classification probabilities or regression confidence intervals are visually displayed or presented with their respective classification results to the user via a display monitor, for example. In another instance, the statistical likelihood or correlations are additionally, or alternatively, visually displayed or presented with their respective classification results to the user via the display monitor.

In another instance, the computer readable instructions cause the processor 106 to display the editable report fields in a particular sequence. For example, in one instance the computer readable instructions cause the processor 106 to first present the editable report fields for anatomical structures with likely abnormal findings and then editable report fields for anatomical structures with likely normal findings. In another example, the computer readable instructions cause the processor 106 to display the report fields in a sequence based on a predetermined template, which can be based on a clinical question/reason for referral. For example, when the referring physician is asking “traumatic knee injury?,” the processor 106 first displays the editable report fields statistically linked to traumatic knee injuries (e.g. ligament ruptures, meniscus tears etc.) so that the user commences with the likely most relevant report fields first. The clinical question/reason can be determined using natural language processing and/or otherwise.

One or more other variations include a combination of one or more of the above variations.

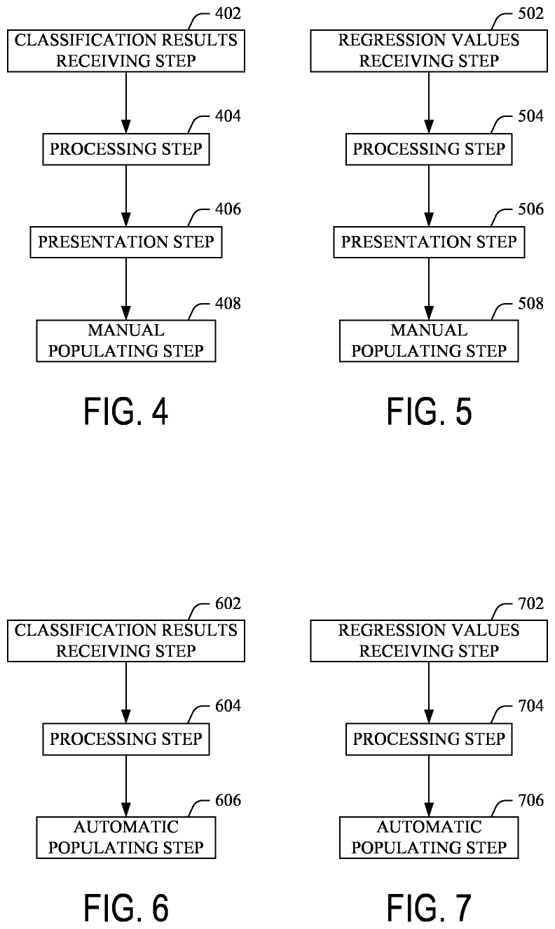

FIGS. 4-8 are directed to computer-implemented methods. It is to be appreciated that the ordering of the acts in one or more of the methods is not limiting. As such, other orderings are contemplated herein. In addition, one or more acts may be omitted, and/or one or more additional acts may be included.

FIG. 4 illustrates an example method for manually populating editable report fields of an electronic structured report where the input includes classification results and classification probabilities, in accordance with an embodiment(s) herein. At a classification results receiving step 402, classification results for at least a subset of editable report fields for an electronic structured report are received as input, as described herein and/or otherwise. At a processing step 404, the classification results are processed based on respective class probabilities, as described herein and/or otherwise. At presentation step 406, the classification results are ranked and displayed, as described herein and/or otherwise. At a manual populating step 408, an editable report field is populated with a classification result selected from the displayed results based on a user input, as described herein and/or otherwise.

FIG. 5 illustrates an example method for manually populating editable report fields of an electronic structured report where the input includes regression values and confidence intervals, in accordance with an embodiment(s) herein. At a regression values receiving step 502, regression values with confidence intervals for at least a subset of editable report fields for an electronic structured report are received as input, as described herein and/or otherwise. At a processing step 504, the regression values are processed based on their respective confidence intervals, as described herein and/or otherwise. At a presentation step 506, the regression values are ranked and displayed, as described herein and/or otherwise. At a manual populating step 508, an editable report field is populated with a regression value selected from the displayed regression values based on a user input, as described herein and/or otherwise. Where the input does not include the confidence intervals, the user can accept the result or reject the result and enter it manually.

FIG. 6 illustrates an example method for automatically populating editable report fields of an electronic structured report where the input includes classification results and classification probabilities, in accordance with an embodiment(s) herein. At a classification results receiving step 602, classification results for at least a subset of editable report fields for an electronic structured report are received as input, as described herein and/or otherwise. At a processing step 604, the classification results are processed based on respective class probabilities, as described herein and/or otherwise. By way of non-limiting example, where the input related to the editable report field for the “Medial meniscus” includes a class “Intact” with a probability of 0.55 (p=0.55), a class “Grade 1 tear” with a probability of 0.25, a class “Grade 2 tear” with a probability of 0.15, and a class “Grade 3 tear” with a probability of 0.05, the processor 106 utilizes the class probabilities to rank/sort the classification results. At an automatic populating step 606, an editable report field is automatically populated with a classification result based on a respective class probability, as described herein and/or otherwise.

FIG. 7 illustrates an example method for automatically populating editable report fields of an electronic structured report where the input includes regression values and confidence intervals, in accordance with an embodiment(s) herein. At a regression values receiving step 702, regression values with confidence intervals for at least a subset of editable report fields for an electronic structured report are received as input, as described herein and/or otherwise. At a processing step 704, regression values are processed based on their respective confidence intervals, as described herein and/or otherwise. At an automatic populating step 706, an editable report field is automatically populated with a regression value based on a respective confidence interval, as described herein and/or otherwise.

The above methods can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not a computer readable storage medium.

While some examples herein have been provided in the field of radiology, the systems, methods and/or operations described herein may be implemented in other areas where structured reporting is used such as cardiology, etc. Furthermore, while some examples herein are described in connection with a knee imaging examination, the systems, methods and/or operations described herein may be implemented for other anatomy and/or organs such as the heart, e.g., in the case of a CVIS or cardiovascular workflow.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

The word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to an advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A computer-implemented method, comprising:
receiving machine learning finding results from an evaluation of imaging data for a subject for at least a subset of editable report fields of an electronic structured report for the subject from a remote device configured to perform the evaluation; and
ranking the machine learning finding results based on predetermined criteria.

2. The computer-implemented method of claim 1, further comprising:
visually displaying the machine learning finding results based on results of the ranking; and
receiving a user input identifying a selected machine learning finding from the displayed machine learning finding results, and, further comprising:
populating an editable report field of the subset of editable report fields based on the user input.

3. The computer-implemented method of claim 1, further comprising:
visually displaying the machine learning finding results using a different visible highlighting for at least two of the machine learning finding results.

4. The computer-implemented method of claim 1, further comprising:
visually displaying only machine learning finding results that satisfy a predetermined threshold, where at least one of the machine learning finding results fails to satisfy the predetermined threshold.

5. The computer-implemented method of claim 1, further comprising:
visually displaying only a user defined predetermined number of the machine learning finding results.

6. The computer-implemented method of claim 5, further comprising:
visually displaying the subset of editable report fields in a predetermined sequence.

7. The computer-implemented method of claim 1, further comprising:
prompting for a user selection and not automatically populating an editable report field of the subset of editable report fields in response to a second editable report field including an abnormal finding result, wherein the editable report field is for a first anatomical structure, the second editable report field is for a second anatomical structure, and the first and second anatomical structures are in close spatial proximity.

8. The computer-implemented method of claim 1, further comprising:
automatically populating an editable report field of the subset of editable report fields with an entry indicating an anatomical structure corresponding to the editable report field is normal in response to the machine learning finding result failing to satisfy a predetermined threshold where machine learning finding results for individual substructures of the anatomical structure satisfy a group predetermined threshold.

9. The computer-implemented method of claim 1, further comprising:
rejecting the machine learning finding results in response to the machine learning finding results failing to satisfy a user-defined entropy threshold.

10. The computer-implemented method of claim 9, wherein the machine learning finding results include classification results with corresponding classification probabilities and the ranking includes ranking the machine learning finding results based on the classification probabilities.

11. The computer-implemented method of claim 1, wherein the machine learning finding results include regression values with corresponding confidence intervals and the ranking includes ranking the machine learning finding results based on the confidence intervals.

12. The computer-implemented method of claim 1, wherein the received machine learning finding results further include statistical results and further ranking the machine learning finding results based on the statistical results.

13. The computer-implemented method of claim 1, further comprising:
automatically populating an editable report field of the subset of editable report fields without a user input selecting a machine learning finding result.

14. The computer-implemented method of claim 13, further comprising:
automatically populating the machine learning finding result only if the machine learning finding result satisfies a predetermined threshold.

15. A system, comprising:
a computer readable storage medium with computer readable instructions; and
a processor configured to:
execute the computer readable instructions, which causes the processor to receive machine learning finding results from an evaluation of imaging data for a subject for at least a subset of editable report fields of an electronic structured report for the subject from a remote device configured to perform the evaluation, and rank the machine learning finding results based on predetermined criteria.

16. The system of claim 15, wherein the machine learning finding results include classification results with corresponding classification probabilities and the ranking includes ranking the machine learning finding results based on the classification probabilities, and wherein the computer readable instructions further cause the processor to visually display the machine learning finding results based on results of the ranking, receive a user input identifying a selected machine learning finding result, and populate an editable report field of the editable report fields based on the user input.

17. The system of claim 15, wherein the machine learning finding results include regression values with corresponding confidence intervals and the ranking includes ranking the machine learning finding results based on the confidence intervals and the machine learning finding results include classification results with corresponding classification probabilities and the ranking includes ranking the machine learning finding results based on the classification probabilities, and further automatically populating an editable report field of the subset of editable report fields without a user input selecting a machine learning finding result.

18. The system of claim 15, wherein the machine learning finding results further include statistical results, and further ranking the machine learning finding results based on the statistical results.

19. A computer readable storage medium storing computer readable instructions, which when executed by a processor of a computer cause the processor to:
receive machine learning finding results from an evaluation of imaging data for a subject for at least a subset of editable report fields of an electronic structured report for the subject from a remote device configured to perform the evaluation,
wherein the machine learning finding results include classification results with corresponding classification probabilities or regression values with corresponding confidence intervals; and
rank the machine learning finding results based on predetermined criteria.

20. The computer readable storage medium of claim 19, wherein the computer readable instructions further cause the processor to populate an editable report field of the editable report fields based on a selected machine learning finding result of the ranked machine learning finding results based on the ranking.
